(19) United States
(12) Patent Application Publication
(10) Pub. No.: US 2013/0303895 A1
(43) Pub. Date: Nov. 14, 2013

(54) System and Method for Performing an Image-Guided Biopsy

(71) Applicant: Delphinus Medical Technologies, Inc., Plymouth, MI (US)

(72) Inventors: Peter John Littrup, Bloomfield Hills, MI (US); Nebojsa Duric, Bloomfield Hills, MI (US); Gerrit Lee Littrup, East Lansing, MI (US)

(73) Assignee: Delphinus Medical Technologies, Inc., Plymouth, MI (US)

(21) Appl. No.: 13/894,202

(22) Filed: May 14, 2013

Related U.S. Application Data
(60) Provisional application No. 61/646,671, filed on May 14, 2012.

(51) Int. Cl. A61B 19/00

(52) U.S. Cl. CPC A61B 19/5244
USPC 600/424

(57) ABSTRACT

A system and method for performing an image-guided biopsy of a target mass of a volume of tissue comprising: a transducer array comprising a set of ultrasound emitters and a set of ultrasound receivers configured to generate a set of acoustic data based upon acoustic waveforms received from the volume of tissue, wherein the transducer array is configured to enable determination of a location of the target mass based on the set of acoustic data; a base proximate to the transducer array; a fixation plate coupled to the base and cooperating with the base and the transducer array to at least partially define an adjustable receiving space configured to receive the volume of tissue; and a guiding module coupled to the base and comprising an aperture configured to align a biopsy tool with the location of the target mass.

### CROSS-REFERENCE TO RELATED APPLICATIONS

This application claims the benefit of U.S. Provisional Application Ser. No. 61/646,671 filed 14 May 2012, which is incorporated in its entirety by this reference.

### TECHNICAL FIELD

This invention relates generally to the medical field, and more specifically to an improved system and method for performing an image-guided biopsy in the medical field.

### BACKGROUND

Breast cancer is the most commonly diagnosed cancer in women and produces the second highest death rate, second only to lung cancer. Many patients undergo breast tissue biopsy during cancer screening processes, which involves removing and analyzing a sample of tissue. Ultrasound technology is a common imaging modality that is used to provide visual guidance when performing a biopsy. However, the quality and accuracy of such guidance using conventional ultrasound technology is highly dependent on scanner quality and operator experience. As a result, performing ultrasound-guided biopsies can be perceived as a daunting task to some radiologists and other medical practitioners. Improved, cost-effective imaging and biopsy techniques are needed to remove operator-dependent uncertainties and improve patient/physician confidence. Thus, there is a need in the medical field to create an improved system and method for performing an image-guided biopsy. This invention provides such an improved system and method for performing an image-guided biopsy.

### BRIEF DESCRIPTION OF THE FIGURES

FIGS. 1A, 1B, and 2 are schematics of the system of a preferred embodiment;

FIGS. 3A and 3B are schematics of the base, transducer array, and fixation plate of the system of a preferred embodiment;

FIGS. 4A-4C are schematics of examples of the system of a preferred embodiment, the examples comprising biopsy image guiding using parallel beams (4A) orthogonal crossing planes (4B), and a coned beam approach (4C);

FIGS. 5A-5B are schematics of variations of the system of a preferred embodiment;

FIGS. 6A-6C are detailed schematics of the guiding module of the system of a preferred embodiment;

FIG. 7 is a schematic of the adjustment of the base, transducer array, and fixation plate of the system of a preferred embodiment; and

FIG. 8 is a flowchart of the processes of the method of a preferred embodiment;

FIGS. 9A and 9B are schematics of a variation of a method of a preferred embodiment;

FIG. 10 is a schematic of a variation of a method of a preferred embodiment; and

FIGS. 11A-11D are exemplary planar images of tissue generated from acoustic data from the system of a preferred embodiment.

### DESCRIPTION OF THE PREFERRED EMBODIMENTS

The following description of preferred embodiments of the invention is not intended to limit the invention to these preferred embodiments, but rather to enable any person skilled in the art to make and use this invention.

### System for Performing an Image-Guided Biopsy

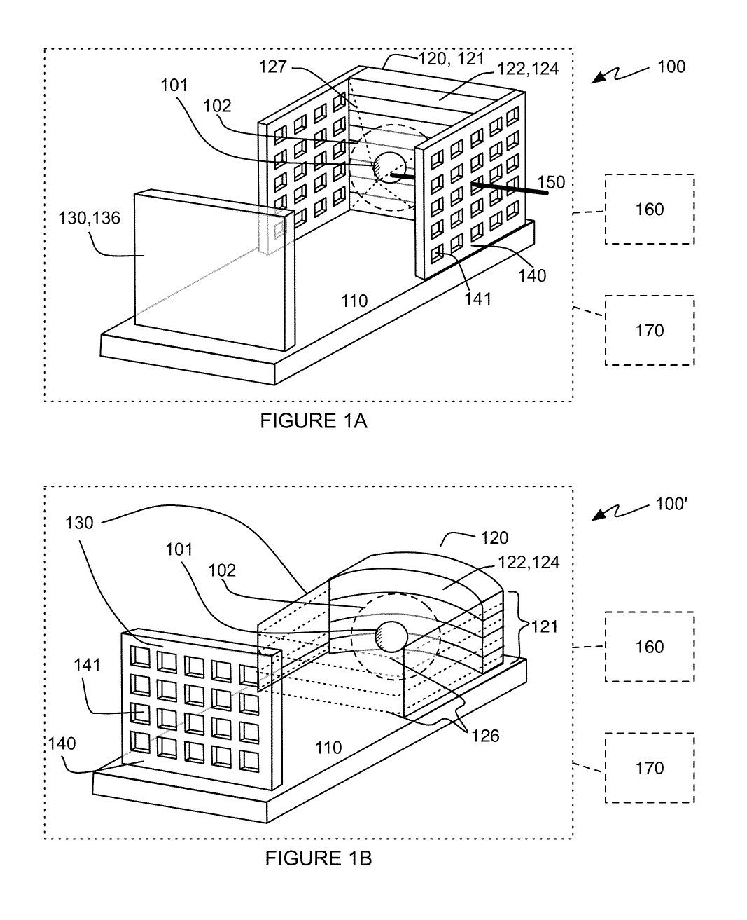

As shown in FIGS. 1A and 1B, an embodiment of a system 100 for performing an image-guided biopsy of a target mass 101 of a volume of tissue 102 includes: a transducer array 120 comprising a set of ultrasound emitters 122 and a set of ultrasound receivers 124 configured to generate a set of acoustic data to enable determination of a location of the target mass 101; a base 110 proximate to the transducer array 120; a fixation plate 130 coupled to the base no and cooperating with the base 110 and the transducer array 120 to at least partially define an adjustable receiving space configured to receive the volume of tissue 102; and a guiding module 140 coupled to the base no and comprising an aperture 141 configured to align a biopsy tool 150 with the location of the target mass 101. The system 100 functions to provide a rapid, easy-to-use, ultrasound-guided biopsy procedure that localizes and/or samples target masses (e.g., suspicious portions) detected by ultrasound tomography. The system 100 can additionally function to facilitate a second-look ultrasound-guided biopsy procedure following suspicious findings after other screening modalities such as magnetic resonance imaging (MRI) or mammograms. The system 100 can be a stand-alone device separate and operationally distinct from an ultrasound tomography scanner or other suitable imaging device, or can be an add-on biopsy solution coupled to an ultrasound tomography scanner or other suitable imaging device. For example, a variation of the system 100 for performing an image-guided biopsy can be an add-on system coupleable to the system described in U.S. Patent Application Publication No. US 2011/0201932, which is incorporated in its entirety by this reference, or another suitable imaging apparatus. However, the system 100 can be independent from another scanning system. The system 100 preferably supports improved participation in breast cancer screening and early detection of breast cancer and identification of other masses (e.g. cyst, fibroadenoma) located in breast tissue. However, the system 100 can additionally or alternatively support biopsy procedures for any suitable kind of tissue, or procedures to obtain samples from any suitable object.

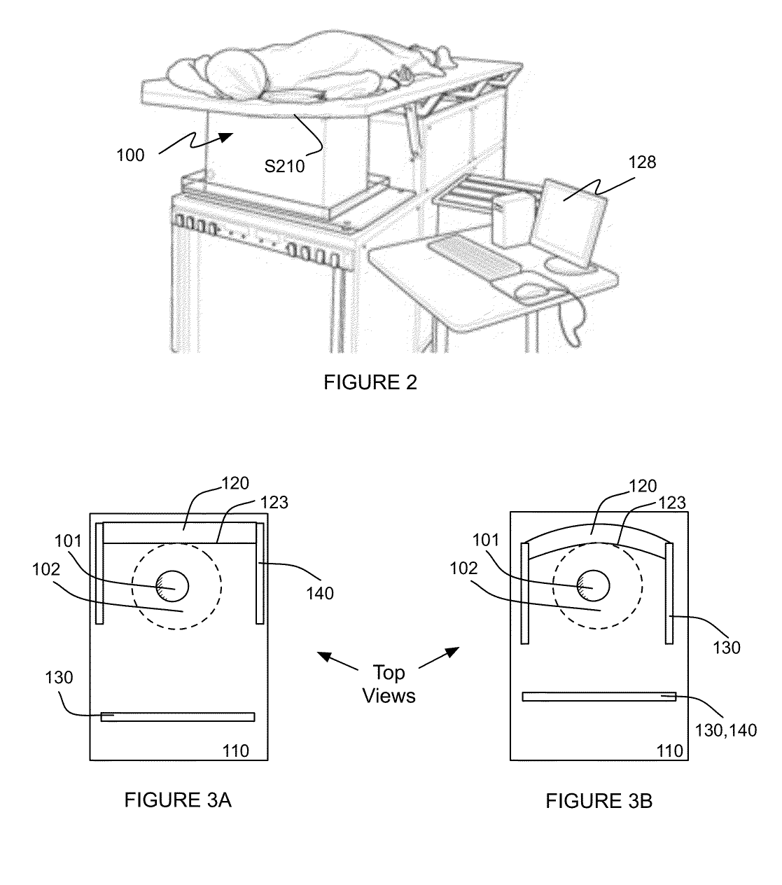

As shown in FIG. 2, in a specific example of the system 100, a patient undergoing the biopsy procedure lies prone stomach-side down on a bed surface located above the preferred system 100. The bed surface in the example defines a hole through which the volume of breast tissue extends. The bed surface, which is made of a durable, flexible material (e.g., sailcloth or another thin membrane), contours to the body of the patient to increase exposure and access to the underlying chest wall and axilla region while maintaining patient comfort. In some variations of the specific example of the system 100, the bed surface can additionally be located above an imaging tank filled with water or another acoustic coupling medium and holding an ultrasound transducer array 120 for enabling ultrasound tomographic scans of the breast tissue. The exposed breast tissue in these variations is preferably pendulous in the air (out of the water in the imaging tank), but can be positioned in any other suitable configuration (e.g., submerged within a medium) in other variations during tomographic scanning and/or during the biopsy procedure.

The transducer array 120 functions to generate data that enables determination of a location of a target mass within the volume of tissue 102, thereby providing guidance for a biopsy procedure. The transducer array 120 preferably generates a set of acoustic data characterizing the interactions between acoustic waveforms and the volume of tissue 102, using a set of ultrasound emitters 122 configured to emit acoustic waveforms toward the volume of tissue 102 and a set of ultrasound receivers 124 configured to receive acoustic signals interacting with the volume of tissue 102. The transducer array 120 can include one or more instances of a single physical transducer element that can function as an ultrasound emitter 122 and an ultrasound receiver 124, and that can be controlled by a switch or other suitable controlling feature to selectively operate in either the transmitting or receiving/detecting mode (e.g., as in some Doppler ultrasound systems). Alternatively, the transducer array 120 can include physically separate ultrasound emitters 122 and ultrasound receivers 124 (e.g., transit-time or transmission ultrasound systems), or any other suitable configuration of ultrasound emitters 122 and ultrasound receivers 124. Furthermore, the ultrasound emitters 122 and receivers 124 can be selectively activated or activated for optimal imaging depending on the application, such as depending on the type or shape of object undergoing a biopsy procedure, or the approximate location of a target mass 101 within a volume of tissue 102 undergoing a biopsy procedure. In variations, the transducer array 120 can include any suitable number of ultrasound emitters 122 and receivers 124 arranged in any suitable configuration and coupled to any suitable element of the system 100.

As shown in FIGS. 3A and 3B, the transducer array 120 can further define a boundary 123 for a volume of tissue undergoing a guided biopsy procedure; however, the transducer array 120 may not define a boundary, but instead may only emit and receive acoustic signals, while another element serves to define a boundary for the volume of tissue. In variations wherein the transducer array 120 defines a boundary 123, the boundary can define a planar surface, as shown in FIG. 3A or a non-planar surface, as shown in FIG. 3B. In these variations, the transducer array 120 can be configured to provide acoustic data that characterizes acoustic reflection within the volume of tissue, or any other suitable acoustomechanical parameter (e.g., acoustic speed, acoustic attenuation) or combination of parameters within the volume of tissue. In one example, a transverse cross-section of the boundary 123 defined by the transducer array 120 can define a segment of a circle or ellipsoid of any suitable shape and/or size (e.g., to define a boundary surrounding or conforming to volumes of tissue of different sizes and shapes). In another example, the boundary 123 can define a curved surface spanning an angle of ˜90 degrees configured to conform to a volume of breast tissue undergoing a biopsy procedure.

The transducer array 120 can also comprise a stack 121 of transducer subarrays configured to provide more than one imaging plane 126, as shown in 1B; however, the transducer array 120 can provide only a single imaging plane or direction, or can comprise a transducer subarray configured to sweep across multiple planes and/or directions (e.g., by beam steering or actuation of the transducer array). Furthermore, each transducer subarray in the stack 121 of transducer subarrays can comprise transducer elements that are arranged in a two-dimensional array of any suitable configuration. For example, any or all transducer subarrays in a stack 121 can comprise transducer elements arranged in a rectangular two-dimensional array.

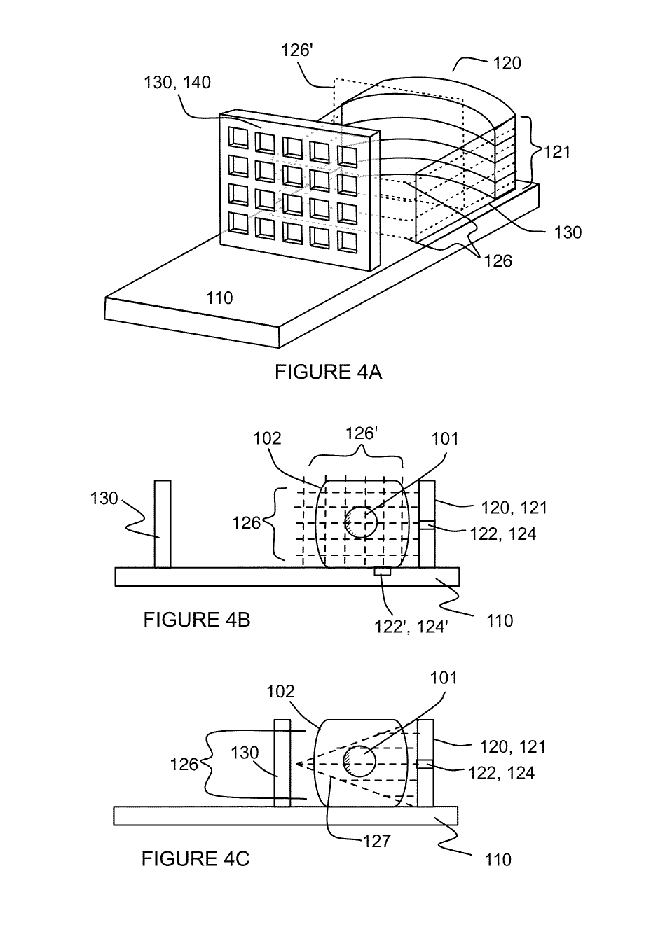

In variations of the transducer array 120 providing more than one imaging plane 126, the imaging planes 126 preferably span an excursion that adequately captures the volume of tissue 102 to be biopsied. For example, in a breast biopsy application, the imaging planes 126 can span a coronal excursion of between 3 and 20 cm to adequately capture a volume of breast tissue. Additionally, in variations of the transducer array 120 providing more than one imaging plane, the imaging planes 126 are preferably parallel to each other; however, the imaging planes 126 can alternatively be oriented in any suitable configuration (e.g., perpendicular, intersecting) that may or may not be adjustable. In one such variation, as shown in FIGS. 4A-4B, the stack 121 of transducer subarrays can provide one or more orthogonal imaging planes 126′, 126, using orthogonally oriented transducer elements 122′, 124′. The orthogonal imaging planes 126, 126′ can be configured to provide acoustic data that characterizes acoustic reflection within the volume of tissue, or any other suitable acoustomechanical parameter (e.g., acoustic speed, acoustic attenuation) or combination of parameters within the volume of tissue. Furthermore, the orthogonal imaging planes 126, 126′ can be enabled using ultrasound elements located at any suitable element(s) (e.g., base, transducer array, fixation plate, guiding module) of the system 100. For example, when used in combination, ultrasound emitters 122 and receivers 124 arranged in a stack 121 of transducer subarrays around the volume of tissue 102 can provide multiple intersecting imaging planes 126, 126′, from which acoustic data can be gathered and analyzed to form various kinds of image renderings (e.g., 2, 2.5, or 3 dimensional renderings) characterizing acoustic reflection within the volume of tissue 102. Additionally or alternatively, transducer subarrays can be configured to be opposed to each other (while surrounding the volume of tissue) to facilitate three dimensional localization of a target mass within the volume of tissue, and to further provide data allowing generation of acoustic transmission images of the volume of tissue.

In another variation, as shown in FIGS. 1A and 4C, multiple imaging planes 126 provided by a stack 121 of transducer subarrays can enable generation of a set of three-dimensional acoustic data, by combining data obtained from multiple two-dimensional imaging planes. The stack 121 of transducer subarrays can additionally or alternatively provide a coned-beam imaging format, whereby signals are emitted and received from any the transducer array 120 elements to generate a three dimensional coned-beam 127 that interacts with a volume of tissue undergoing a biopsy procedure. The coned-beam imaging format can enable generation of a coned-beam 127 of any suitable dimensions (e.g., height, width, diameter) or profile (e.g., pyramidal, conical). Furthermore, the volumetric coned-beam approach can provide imaging planes only through a target mass of the volume of tissue upon rescanning (i.e., re-slicing) the volume of tissue. Furthermore, an ultrasound beam generated by the transducer array 120 or by a subset of a stack 121 of transducer subarrays can be adjustable in dimensions or profile, such that ultrasound echoes from a subset of transducer elements can be received by other transducer elements of the transducer array 120.

In a first specific example of the transducer array 120, as shown in FIG. 1A, the transducer array comprises a stack 121 of eight transducer subarrays configured to form a wall with a planar surface. In the first specific example, ultrasound signals emitted by and/or received from any of the eight transducer subarrays function to generate a three-dimensional coned beam 127 that interacts with a volume of tissue undergoing a biopsy procedure. Each of the eight transducer subarrays comprises a two dimensional rectangular array of transducer elements (functioning both as emitters and receivers) arranged in a 4×256 element grid; thus, the transducer array 120 in the first specific example comprises a total of 8192 transducer elements, and each of the eight transducer subarrays comprises 1024 transducer elements. Signals received from multiple imaging planes by the transducer array 120 in the first specific example can be used to generate a set of three-dimensional acoustic data that enables identification of a location (in three-dimensional space) of a target mass within the volume of tissue.

In a second specific example of the transducer array 120, as shown in FIG. 1B, the transducer array 120 includes a stack 121 of four transducer subarrays, configured to form a curved boundary 123, such that the transducer array 120 provides multiple primary scanning planes and conforms to a surface of a volume of tissue. The depth or thickness of each scanning plane in the second specific example is adjustable depending upon the width of an acoustic beam emitted by the transducer array 120. In a variation of the second specific example, the transducer array 120 can include modular arc segment elements that can be arranged contiguously to form an enclosed ring, as described in U.S. Patent Application Publication No. US 2011/0201932, which is incorporated in its entirety by this reference. In another variation of the second specific example, module arc segments can be arranged contiguously and stacked to form a multi-level arc segment (or alternatively, arranged contiguously in one plane to form a single-level arc segment).

As shown in FIGS. 1A and 1B, the transducer array 120 is preferably configured to communicate with a processor 170, wherein the processor 170 is configured to process a set of acoustic data from the transducer array 120. The processor 170 thus functions to receive a set of acoustic data from the transducer array 120, and to enable detection and determination of a location of a target mass 101 within the volume of tissue 102, based upon the set of acoustic data. The processor 170 is preferably configured to simultaneously process acoustic data from multiple imaging planes 126, to process acoustic data from multiple transducer subarrays (e.g., modular subarrays), and to process acoustic data generated using a coned beam. To achieve these functions, the processor 170 preferably comprises a data acquisition module configured to process multiple transmit and receive channels, and at least one multiplexer configured to aggregate multiple input signals and/or output signals. As such, the processor 170 is preferably configured to render an image “slice” from a single plane, a “2.5 dimensional” image, or a three-dimensional image based on acoustic data from multiple imaging planes, such that a location of a target mass 101 within the rendering can be determined to provide biopsy guidance. The rendering can be presented on a display 128 of a user interface, and can characterize a distribution of one or a combination of acoustomechanical properties, such as acoustic reflection, acoustic speed, and acoustic attenuation. The processor 170 may be the processor described in U.S. application Ser. No. 13/756,851, entitled “System and Method for Imaging a Volume of Tissue”, which is incorporated in its entirety by this reference, or may be any other suitable processor 170 configured to determine a location of a target mass 101 within a volume of tissue 102.

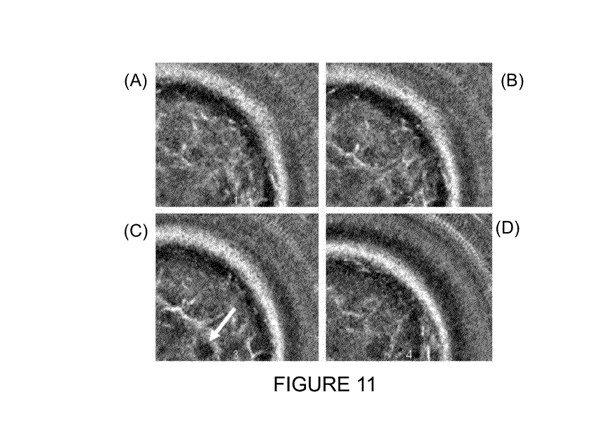

In an example transducer array/processor interaction, the transducer array 120 generates a set of acoustic data that is received and processed by a processor 170 to provide reflectivity data on acoustic signals reflecting off the surface of and within a volume of tissue 102 to be biopsied, such that real-time or near real-time reflection ultrasound images can be constructed from the reflection data and rendered on a display 128. The acoustic data and/or ultrasound images in the example enable detection of target masses with a dimension (e.g., diameter) greater than 5 mm, such that a location of detected target masses can be determined. To achieve this, the transducer array 120 and the processor 170 in the example communicate to generate cross-sectional “slices” of acoustic reflection images based upon acoustic data gathered within a respective imaging plane 126. A portion of these cross-sectional slices can image the targeted mass more clearly than other cross-sectional slices depending on degree of alignment of the targeted mass within the imaging plane, as shown in FIGS. 11A-11D, which can be used to adjust or calibrate alignment of the transducer array 120. The transducer array 120 in the example can additionally or alternatively provide acoustic data representing other interactions between the acoustic signals and the volume of tissue 102 (or other irradiated object), such as to provide measurements of acoustic attenuation based upon amplitude changes of acoustic waves in the tissue, acoustic speed based on departure and arrival times of acoustic signals between emitter-receiver pairs, and/or any suitable acoustic parameter that can analyzed to develop an image of the targeted mass within the volume of tissue 102.

In another example transducer array/processor interaction, the system 100 includes eight modular transducer arrays, each consisting of 256 separate transducer elements to generate a total of 2048 data channels or sets. The extensive processing capacity of this current ultrasound tomography embodiment allows rapid data transfer and image reconstruction within 15 minutes for both breasts, with a processor 170 comprising multiple parallel graphic processing units (GPUs) and computer processing units (CPUs). The example configuration of the transducer array 120 and processor 170 thus allows any combination of multiple transducer arrays to surround a tissue volume to appropriately detect targets or masses within a volume of tissue, with simultaneous detection from all eight or more arrays. Storage of the associated images from the multiple arrays then allows processing of the multiplanar data, at the processor 170, to generate a three-dimensional representation of the tissue volume for guidance targeting. Cartesian or polar coordinates, or any other three-dimensional mapping of location coordinates, can thus be used him to better define the optimum needle path or trajectory from skin surface to the mass within the image volume. This can also include, but is not limited to, matching of three-dimensional images from prior imaging studies (i.e., CT, breast MR, UST-in-water) using any manner of localization matching of anatomic sites to target masses, such as software morphing.

As shown in FIGS. 1A-1B and 4A-4C, the base no of an embodiment of the system 100 is preferably proximate to the transducer array 120, and functions to receive and support a volume of tissue 102. As shown in FIG. 1, the base 110 is preferably a substantially planar fixation plate 130, but can be of any suitable shape to receive and support a volume of tissue 102. Additionally, the base 110 is preferably coupled to the transducer array 120, such that a surface of the transducer array 120 is substantially orthogonal to a surface of the base 110. In this configuration, at least one imaging plane 126 provided by the transducer array 120 is substantially parallel to the base 110. The base can, however, be arranged in any other suitable configuration relative to the transducer array 120 and can comprise transducer elements to provide orthogonal imaging planes, as described above.

The base 110 can be coupled to one or more actuators (e.g. stepper motor) that function to reposition the base 110 along an anterior-posterior direction relative to the patient (e.g., vertical with respect to the prone patient on a bed surface) and/or along any suitable axis (e.g., medial-lateral, inferior-superior). Alternatively, the base 110 can be manually adjustable to align a volume of tissue 102 undergoing a biopsy procedure. In one example, the base no is movable to gently lift a pendulous breast to be aligned relative to the transducer array 120, in order to confine the axial breast length (as defined along the anterior-posterior direction) to the estimated height or suitable other scan area of the transducer array 120. Furthermore, the breast tissue can be confined to substantially or approximately match a configuration of the breast tissue taken in a previous scan of the tissue (e.g., such as the breast hanging excursion from an initial ultrasound tomographic scan or during magnetic resonance imaging) in order to better capture the location of a target mass in the volume of tissue in 3D space. However, in some applications it can be sufficient to move the base no to support and lift the volume of tissue 102 such that at least the target mass (e.g., as identified prior to biopsy by ultrasound tomography or other imaging methods) is within the scan region of the transducer array 120, regardless of whether the entire volume of tissue 102 is within the scan region of the transducer array 120.

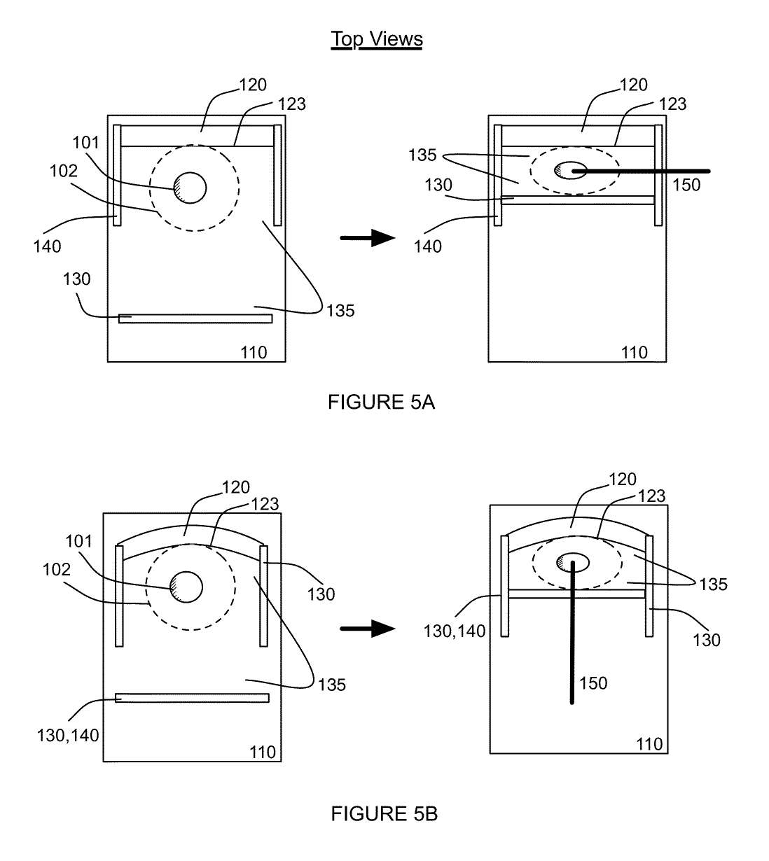

The fixation plate 130 of the system 100 is preferably coupled to the base, and functions to at least partially define an adjustable receiving space 135 configured to receive the volume of tissue 102. As shown in FIGS. 1A-1B and 5A-5B, the fixation plate 130 is preferably approximately planar, but can alternatively be concave or any suitable shape. In one alternative variation, the fixation plate 130 can comprise a non-planar surface configured to conform to a volume of tissue. The fixation plate 130 preferably includes at least a portion that is directly opposite the transducer array 120, as shown in FIGS. 5A and 5B. Additionally, the fixation plate 130 is preferably movable along the base no to adjust the size of the receiving space 135, such that selective positioning of the fixation plate 130 can compress the volume of tissue 102 within the receiving space 135, in cooperation with the base no and/or the transducer array 120. The transducer array 120 is preferably in a fixed position relative to the base no, and the fixation plate 130 is movable relative to the base no to compress the received volume of tissue 102 against the base no and/or transducer array 120. Alternatively, the fixation plate 130 can be in a fixed position relative to the base no, and the transducer array 120 can be movable to compress the received volume of tissue 102 against the base no and/or fixation plate 130. In another alternative, the fixation plate 130 and the transducer array 120 can both be movable toward one another to compress the volume of tissue 102. In any of these variations, the fixation plate, the transducer array 120, and/or the base no can move relative to one another along a continuum in any suitable direction (e.g., linearly, radially) such as with a system of slots, tracks, belts, wheels, or other suitable adjustable mechanisms, and/or along a series of discrete positions. However, the base no, transducer array 120, fixation plate 130, and/or any suitable components can function to receive and/or compress the volume of tissue 102 in any suitable manner.

The fixation plate 130 can further function to reflect acoustic signals, in order to facilitate generation and assessment of acoustic speed and/or acoustic attenuation data. As such, the fixation plate 130 can be coupled to a reflector plate 136, can be physically coextensive with a reflector plate 136, can be of unitary construction with a reflector plate 136, or can be a reflector plate 136. In variations wherein the fixation plate 130 functions to reflect acoustic signals, the fixation plate 130 is preferably opposite the transducer array 120; however, the fixation plate 130 can be oriented in any suitable configuration relative to the transducer array 120. Furthermore, the fixation plate 130 and/or reflector plate 136 can be adjustable in position relative to other elements of the system 100, as described above. Moreover, the fixation plate 130 can comprise an opposing transducer array (i.e., a transducer array opposing the transducer array 120) in order to have direct transmission imaging characteristics enabling analyses of sound speed and attenuation parameters. As such, the opposing transducer arrays (e.g., on opposed faces of a volume of breast tissue) can comprise planar or non-planar (e.g., curved) surfaces, with non-planar surfaces further facilitating reconstruction algorithms and imaging. In a specific example, as shown in FIG. 1A, the fixation plate 130 is a reflector plate 136 defining a surface that is parallel to a surface of the transducer array 120, wherein both the surface of the fixation plate 130 and the surface of the transducer array 120 are orthogonal to a surface of the base 110.

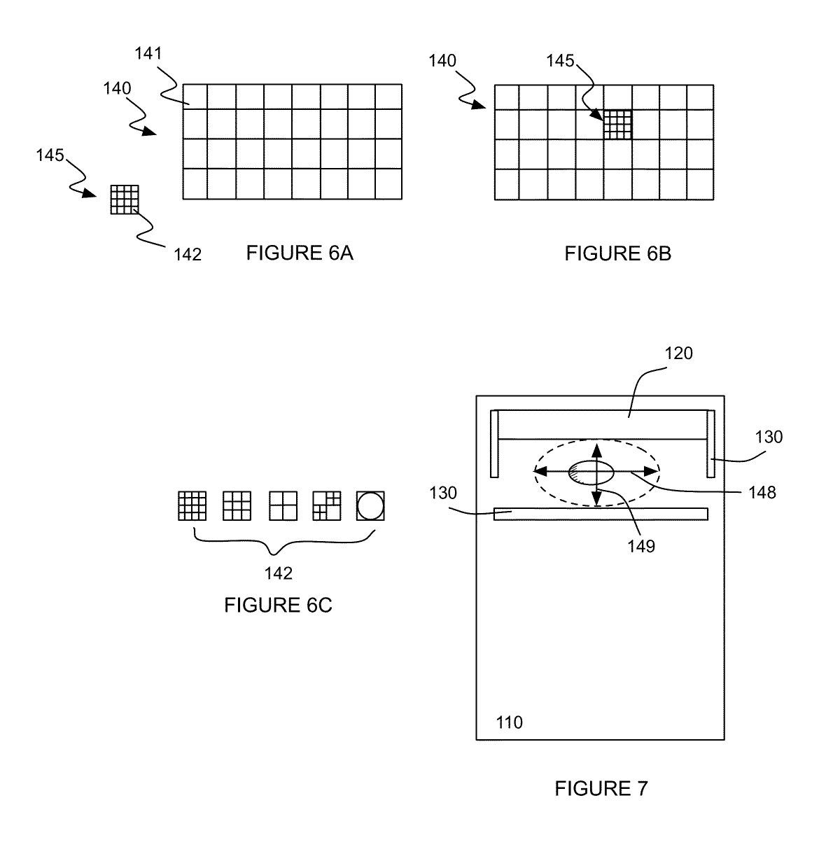

The guiding module 140 preferably defines a series of apertures 141 through which a biopsy tool 150 can pass to access the received volume of tissue 102 and/or target mass 101. The guiding module 140 can be coupled to the base no, or to any other suitable element of the system 100 or external to the system 100. In particular, as shown in FIG. 6A, the series of apertures 141 is preferably arranged in a grid, and each aperture 141 is preferably configured to receive an insert 145 configured to receive and align a specific biopsy tool 150 relative to the volume of tissue 102 and/or target mass 101. In an example, the insert 145 can be a needle guide insert defining passageways 142 of suitable diameter for one or more needle gauges (sizes), such that the biopsy tool 150 is a biopsy needle. The apertures 141 can include rectangular cutouts arranged in a rectangular grid, but can additionally or alternatively include apertures of any suitable shape, suitable size, or in any suitable arrangement, such that any location within the volume of tissue to be biopsied can be accessed by the biopsy tool 150 through an aperture 141. In another variation, the guiding module 140 can additionally or alternatively define suitable passageways for one or more different biopsy tools 150 (e.g., different in size, profile, etc.), separate from an insert 145. Furthermore, in other variations, the guiding module 140 can be coupled to an actuator or can be otherwise movable, such that an aperture 141 of the guiding module 140 can be moved relative to a received volume of tissue 102. Other variations of the guiding module 140 can include a modified grid containing multiple apertures 141, such that portions of the grid are solid and contain ultrasound transducers or reflect acoustic signals. In variations of the guiding module 140, these ultrasound transducers can be positioned within every other aperture 141 of the guiding module 140 or in any other suitable arrangement. Resulting acoustic data could thus provide potential transmission parameters (e.g., acoustic speed, acoustic attenuation) by sending and/or receiving ultrasound signals between the guiding module 140 and the overall transducer array 120.

In one variation, the guiding module 140 can be oriented transversely in relation to the transducer array 120 and/or fixation plate 130, as shown in FIGS. 1A 5A, and 7, such that a biopsy tool 150 can be guided into the volume of tissue 102 using a long axis 148 of tissue stabilization approach. In another variation, as shown in FIGS. 1B, 5B, and 7, the guiding module 140 can be oriented with a surface substantially opposite a surface of the transducer array 120 and/or fixation plate 130, such that a biopsy tool 150 can be guided into the volume of tissue 102 using a short axis 149 of tissue stabilization approach. In another variation, the guiding module 140 can substantially frame three sides of volume of tissue (e.g., at least a portion of the guiding module 140 can be coupled to, physically coextensive with, or of unitary construction with the fixation plate), in order to provide a configuration that allows the volume of tissue to be accessed from three sides (e.g., spanning a 270 degree angle) during a biopsy procedure. In another variation, the guiding module 140 can be oriented in any suitable configuration relative to the transducer array 120 and/or fixation plate 130, such that a biopsy tool 150 can be guided into the volume of tissue 102 along any direction. Furthermore, in one variation, the guiding module may be coupled to (e.g., physically coextensive with or of unitary construction with) the fixation plate 130, as shown in FIGS. 1B and 5B, or may not be coupled to the fixation plate 130, as shown in FIGS. 1A and 5A. In other variations, the guiding module 140 can be movable (e.g. manually, by actuation) or can be fixed relative to other elements of the system 100.

The insert 145 functions to align a biopsy tool 150 with the target mass 101 of the volume of tissue 102 after a location of the target mass 101 or other feature of interest has been determined. As shown in FIG. 6B, the insert 145 preferably couples to an aperture 141 or other suitable receptacle in the guiding module 140. For example, the insert 145 can snap-fit into a framework surrounding the aperture 141, or couple to the guiding module 140 in any suitable manner. Alternatively, the insert 145 can couple to a second guiding module or other structure adjacent to the first guiding module 140. The insert 145 preferably includes an array of passageways of suitable dimensions for one or more biopsy tools 150. The insert 145 can also be one selected from a plurality of inserts of assorted sizes, depending upon the intended biopsy tool 150. For example, the system 100 can include a set of assorted needle guide inserts, each defining needle guide passageways 142 of a particular size, as shown in FIG. 6C. In another example, each insert 145 can define a plurality of needle guide passageways 142 of various sizes. In the examples, the needle guide passageways 142 can be configured to receive biopsy tools 150 ranging from a fine needle for hookwire placement in the targeted mass to an eight-gauge needle for vacuum-assisted biopsy (VAB) devices for percutaneous biopsy, or can have any suitable dimensions for any suitable size of needle or other biopsy tool 150. In one variation, at least one of the passageways is configured to guide a biopsy tool 150 in a direction perpendicular to the face of the insert 145. In another variation, at least one of the passageways is non-perpendicularly angled relative to the face of the needle guide insert 140 (e.g., up to 45 degrees), providing needle guidance for more biopsies in more difficult locations, such as near the surface of the volume of tissue (e.g., chest wall) or near a given tissue feature (e.g., nipple).

In other embodiments of the system 100, the guiding module 140 may not comprise apertures 141 and/or may not be coupleable to an insert 145. For instance, the guiding module 140 can comprise a pillar and post guiding module, a stereotaxic guiding module (e.g., framed or frameless), or any other suitable guiding module 140.

As shown in FIGS. 1A and 1B, the system 100 can further comprise or be coupleable to a control system 160, which preferably drives the individual transducer elements to send and/or receive an ultrasound signal. The control system 160 can also be used to process the data as previously described by algorithms of U.S. Patent Application Publication No. US 2011/0201932, which is incorporated in its entirety by this reference. The control system 160 functions to at least control the transducer array 120 and its ultrasound emitters 122 and detectors 124 (e.g., activation of emitters 122 and detectors 124), and/or actuation of the base 110, transducer array 120, fixation plate 130, reflector plate 136, and/or guiding module 140. The control system 160 can further function to guide a biopsy tool 150 into a volume of tissue upon determination of the location of a target mass 101 by the processor 170. The control system 160 can, however, by any suitable control system 160.

Various embodiments of the system 100 can include any combination of the base 110, transducer array 120 and other transducer elements, fixation plate 130, and guiding module 140. Furthermore, the positions of any combination of the base 110, transducer array 120, fixation plate 130, and/or any other suitable component can be positionable in any suitable manner to properly align the volume of tissue 102 (or at least the targeted mass) with at least one imaging plane provided by the transducer array 120. Additionally, at least a portion of the components, such as the base 110 and the fixation plate 130, can be made of injected molded plastic. However, these components can alternatively machined or otherwise formed from any suitable material.

As a person skilled in the art will recognize from the previous detailed description and from the FIGURES and claims, modifications and changes can be made to the preferred embodiments of the system 100 without departing from the scope of the system 100.

### Method for Performing an Image-Guided Biopsy

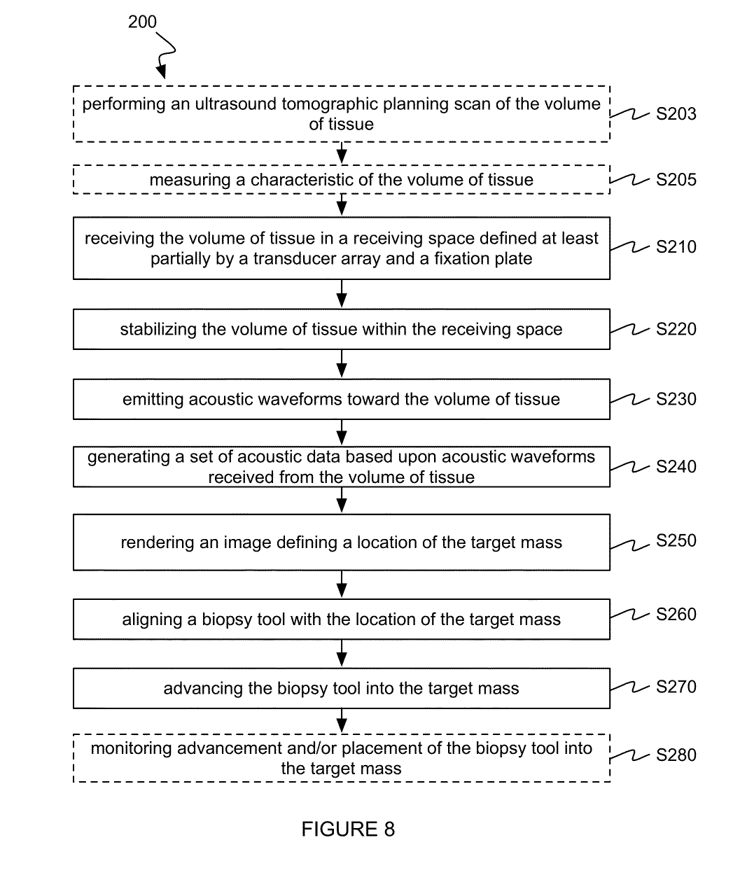

As shown in FIG. 8, an embodiment of a method 200 for performing an image-guided biopsy of a target mass of volume of tissue comprises: in Step S210, receiving the volume of tissue in a receiving space defined at least partially by a transducer array and a fixation plate; in Step S220, stabilizing the volume of tissue within the receiving space; in Step S230, emitting acoustic waveforms toward the volume of tissue; in Step S240, generating a set of acoustic data based upon acoustic waveforms received from the volume of tissue; in Step S250, rendering an image defining a location of the target mass, based upon the set of acoustic data; in Step S260, aligning a biopsy tool with the location of the target mass; and in Step S270 advancing the biopsy tool into the target mass.

The method 200 can provide a rapid, ultrasound-guided biopsy procedure that localizes targeted masses (e.g., suspicious portions) detected by ultrasound tomography and/or provides an second-look ultrasound-guided biopsy procedure following suspicious findings after other screening modalities such as magnetic resonance imaging (MRI) or mammograms. The method 200 can further function to reduce operator dependence when performing ultrasound-guided biopsies. The method 200 is preferably performed in conjunction with ultrasound tomography to identify suspicious portions of tissue prior to biopsy, but can additionally or alternatively be performed in conjunction with other imaging processes, or independently of other imaging modalities. The method 200 can support improved participation in breast cancer screening and early detection of breast cancer and identification of other masses (e.g. cyst, fibroadenoma) located in breast tissue. However, the method 200 can additionally or alternatively support biopsy procedures for any suitable kind of tissue, or procedures to obtain samples from any suitable object.

In one embodiment of the method 200, as shown in FIG. 8, the method 200 can further include performing an ultrasound tomographic planning scan of the volume of tissue in Step S203, and measuring a characteristic of the volume of tissue in Step S205. Steps S203 and S205 are preferably performed prior to the biopsy, but can be performed at any stage of the method 200.

Step S203 recites performing an ultrasound tomographic planning scan of the volume of tissue, and functions to provide an initial scan of the volume of tissue (e.g., to determine the existence of a suspicious mass and to determine whether a biopsy is advisable) and/or to identify the location of a suspicious mass. Step S203 can comprise manipulating the volume of tissue into a stabilized configuration, or can comprise manipulating the volume of tissue in any suitable manner. Performing an ultrasound tomographic scan in Step S203 is preferably similar to that described in U.S. Patent Application Publication No. US 2011/0201932, and can be performed with the volume of tissue submerged in a fluid-filled imaging tank. In particular, the ultrasound tomographic scan can capture renderings based on acoustic data representing the interaction between acoustic waves and the volume of tissue in terms of acoustic reflection, acoustic attenuation, acoustic speed, any other suitable acoustic parameter (e.g., elasticity), and/or any combination of parameters. The ultrasound tomographic scan and the imaging of the tissue during the biopsy can be performed by the same set of modular transducer elements, such that the modular transducer elements are reconfigurable for multiple purposes, or can be performed by different transducer elements.

Step S205 recites measuring a characteristic of the volume of tissue, and functions to provide a characteristic measurement that is representative of the shape of the volume of tissue. Step S205 can comprise measuring a characteristic of the volume of tissue that has been manipulated to a stabilized configuration in a variation of Step S203. Step S205 can also be performed for a volume of tissue submerged in a fluid-filled imaging tank. For example, when the volume of tissue is outside of the fluid-filled imaging tank and stabilized during the biopsy procedure, the measured characteristic is can be used as a benchmark to verify that the shape of the volume of tissue approximates that of the submerged volume of tissue. The characteristic can include a measurement of any suitable dimension or parameter of a volume of tissue, such as a measurement of the width, length, diameter, or volume of the volume of tissue. In one specific application, the measured characteristic can include the axial length of the pendulous breast in the prone position (that is, a length of the breast volume along the anterior-posterior direction).

Step S210 recites receiving the volume of tissue in a receiving space defined at least partially by a transducer array and a fixation plate, and functions to place the volume of tissue proximate to the ultrasound transducer array and in position for a biopsy procedure. In variations of Step S210, the receiving space can be defined by any suitable element(s) of the system described above, such as the transducer array 120, the base 110, the fixation plate 130, and/or the guiding module 140. As shown in FIG. 2, in one specific example of Step S210, a patient undergoing the biopsy procedure lies prone stomach-side down on a bed surface located above the receiving space. The bed surface preferably defines a hole through which the volume of breast tissue preferably extends. The bed surface, which can be made of a durable, flexible material such as sailcloth or another thin membrane, preferably contours to the body of the patient, thereby increasing exposure and access to the underlying chest wall and axilla region, while maintaining patient comfort. During the biopsy procedure, the exposed breast tissue in the specific example is pendulous in the air (out of the water in the imaging tank) and received in the receiving space in Step S210. In other variations of Step S210, the volume of tissue can be received in a receiving space defined at by any other suitable element(s).

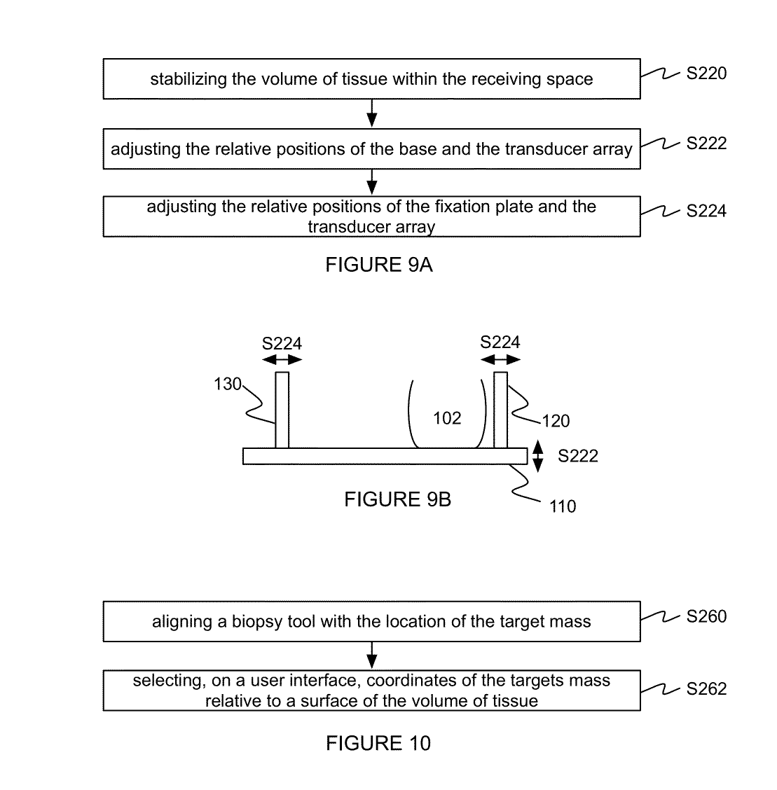

Step S220 recites stabilizing the volume of tissue within the receiving space, and functions to secure the volume of tissue in place. Step S220 can further function to manipulate the volume of tissue into a shape that approximates that of the stabilized configuration of the volume of tissue defined in variations of Steps S203 and/or S205, such that the information related to the location of the targeted mass within the tissue, as determined during a prior tomographic or other imaging scan of the tissue, is applicable to the volume of tissue during the biopsy procedure. In variations using the system 100 described above, as shown in FIGS. 9A and 9B, Step S220 can further include at least one of: adjusting the relative positions of the base and the transducer array in block S222, and adjusting the relative positions of the fixation plate and the transducer array in block S224. Alternatively, since stabilization of the volume of tissue involves the relative distances between at least two of the base, the transducer array, and the fixation plate in these variations, Step S220 can be described in terms of adjusting the positions of any of the base, transducer array, and fixation plate relative to one another.

Adjusting the relative positions of the base and the transducer array in Step S222 functions to confine the volume of tissue (or at least the targeted mass) to the scan region of the transducer array and/or to manipulate the volume of tissue to approximate the shape and internal mass location of the stabilized configuration of the volume of tissue. In one variation of Step S222 using a variation of the system 100 described above, the base is actuated vertically relative to the transducer array, supporting the volume of tissue at a suitable tissue surface (e.g., along an anterior-posterior direction, inferior-superior direction, or medial-lateral direction of the patient), until a defining tissue dimension (e.g., axial length) equals the defining tissue dimension measured on the previously analyzed volume of tissue (e.g., submerged volume of tissue in Step S203 or Step S205). In another variation of Step S224, the transducer array is additionally or alternatively actuated vertically relative to the base while the base supports the volume of tissue from at any suitable tissue surface. In both variations, the final relative positions of the base and the transducer array preferably depend on the particular dimension or other characteristic measured in Step S205.

Adjusting the relative positions of the fixation plate and the transducer array in Step S224 can further function to stabilize the volume of tissue within the receiving space. In one variation of Step S224, the fixation plate and/or the transducer array are actuated to reduce a distance between the fixation plate and the transducer array, such as along the base using tracks, slots, or other guidance mechanisms. In another variation of block S224, the transducer array and/or the fixation plate can be additionally or alternatively actuated to reduce a distance between the fixation plate and the transducer array, such as along the base similar to the first variation. In both variations, the final relative positions of the fixation plate and the transducer array preferably compress the volume of tissue enough to stabilize the tissue against the insertion of a biopsy tool, and can further substantially manipulate the volume of tissue to approximate the characteristics (e.g., size, shape and/or internal contents) determined in Step S205.

Step S230 recites emitting acoustic waveforms toward the volume of tissue, and functions to provide waveforms that interact with the volume of tissue, such that a set of acoustic data characterizing the location of a target mass in the volume of tissue can be determined. Emitting acoustic waveforms toward the volume of tissue is preferably performed at the transducer array of a variation of the system 100 described above, but can be performed using any suitable element configured to emit acoustic waveforms toward a volume of tissue. Step S230 can comprise emitting acoustic waveforms using an ultrasound transducer array simultaneously providing multiple imaging planes, as described above, and can alternatively or additionally comprise emitting acoustic waveforms characterized by a three-dimensional coned-beam format. Step 230 can alternatively comprise emitting acoustic waveforms within a single imaging plane or within an imaging plane configured to sweep along an excursion path spanning a portion of interest of the volume of tissue (e.g., whole tissue volume or quadrant of a tissue volume). Step S230 can also be performed in conjunction with another imaging modality (e.g., computed tomography, coherence tomography, resonance imaging, etc.) such that a location of a target mass can be verified. Opposing arrays in either individual or multiple stacks may also be used to generate reflection imaging from opposed surfaces of a volume of tissue (e.g., sides of a breast), as well as transmission imaging to achieve direct sound speed and attenuation data.

Step S240 recites generating a set of acoustic data based upon acoustic waveforms received from the volume of tissue, and functions to provide data that can be used to determine a location of a target mass within the volume of tissue. Step S240 can further function to provide data that can be used to render an image of the target mass and/or volume of tissue. Step S240 can thus comprise receiving the acoustic waveforms at a transducer array comprising a set of ultrasound receivers, and in one variation, comprises receiving acoustic waveforms at a variation of the transducer array described above. Step S240 can alternatively comprise receiving acoustic waveforms using any other suitable element. The set of acoustic data preferably comprises data obtained from multiple imaging planes (e.g., parallel, orthogonal, intersecting) using a coned-beam imaging format, but can alternatively comprise data obtained from a single imaging plane using any suitable beam format.

Step S250 recites rendering an image defining a location of the target mass based upon the set of acoustic data, and functions to enable visual guidance of the location of the target mass within the volume of tissue. In one variation, Step S250 is preferably performed at a variation of the processor described above, and preferably provides a real-time or near real-time image of the volume of tissue based on acoustic data gathered from multi-level imaging planes intersecting the stabilized volume of tissue. The image of the volume of tissue and/or location of the target mass is preferably at least a “2.5”-dimensional image (and preferably a three-dimensional image) based upon the set of acoustic data generated in Step S240, but can be any suitable image at any suitable resolution that enables determination of a location of a target mass. In a specific example, the image is a 2.5-dimensional rendered at a resolution of 14 bits, with a reconstitution time of approximately 4 seconds to provide a near real-time image. Step S250 preferably includes rendering an image of the volume of tissue based upon acoustic reflection data, but can additionally or alternatively include imaging the volume based on any suitable acoustic parameter (e.g., acoustic speed, acoustic attenuation, elasticity) for additional mass localization capability. Step S250 preferably includes rendering the image of the volume of tissue on a display (e.g., monitor), such that an operator (e.g., medical practitioner) can view the image and determine, with higher accuracy and confidence, the location of a target mass within the volume of tissue.

Step S260 recites aligning a biopsy tool with the location of the target mass, and functions to position the biopsy tool proximate to the target mass. Step S260 preferably uses near real-time imaging (e.g., acoustic reflection imaging) for direct visualization and alignment. Additionally or alternatively, additional transmission parameters can be overlaid or fused upon the reflection images, either by co-localization with prior three-dimensional planning scans (e.g., breast MR, ultrasound tomography in-water), and/or directly obtained from processing transmission data between the transducer array and a fixation plate/reflector plate, or a set of opposed transducer arrays within a plate or curved array architecture to directly measure sound speed and attenuation data. Step S260 can include selecting a suitable insert and coupling the insert to an aperture of a guiding module, as described in variations of the system 100 described above. For example, Step S260 can comprise selecting a needle guide insert defining needle guide passageways corresponding to the gauge (size) of the biopsy needle and coupling the selected needle guide insert to the an aperture of a guiding module. In the example of Step S260, the needle guide insert can be selected from a plurality of available needle guide inserts configured to receive biopsy tools 150 ranging from a fine needle for hookwire placement in the targeted mass to an eight-gauge needle for vacuum-assisted biopsy (VAB) devices for percutaneous biopsy, or any other suitable biopsy tool. Step S260 can further include defining an angle of alignment (e.g., orthogonal to a tissue surface or at an angle relative to a tissue surface) using a suitable guiding module, in order to facilitate specialized approaches for biopsies in more difficult locations such as near the chest wall, nipple, skin, or other tissue feature.

As shown in FIG. 10, some embodiments of the method 200 can include Step S262, which recites selecting, on a user interface, coordinates of the targeted mass relative to a surface of the volume of tissue (e.g., skin surface). Step S262 can comprise using localization and biopsy guidance software that define guidance options for a projected path from the skin surface to the target mass. Step S262 preferably uses reflection data obtained from the multiplanar biopsy array configurations, but can additionally or alternatively utilize superimposed, or fused, data from either breast CT, MR, or ultrasound tomography. In some variations, Step S262 can comprise utilizing a user interface that allows interaction with the images in the localization software to select the 3-D coordinates of the internal mass/target location in relation to a suitable reference (e.g., tissue surface). The intended path or trajectory can further enable determination of an appropriate aperture, insert, and/or biopsy tool combination that is most appropriate for the biopsy procedure.

Step S270 recites advancing the biopsy tool into the target mass, and functions to obtain a biological sample of the targeted mass, position a marker in the targeted mass, and/or otherwise interact with the targeted mass using the biopsy tool. Step S270 can be performed manually by an operator, or can be performed automatically using an actuation system coupled to a control system. Furthermore, Step S270 can comprise advancing the biopsy tool along either a long axis of tissue stabilization, a short axis of tissue stabilization, or along any suitable direction. In one example, Step S270 can include placing a hookwire into or through the targeted mass, performing an automatic core biopsy, performing a vacuum-assisted biopsy, and/or any suitable biopsy procedure. Step S270 can further include anesthetizing at least a portion of the volume of tissue prior to advancing the biopsy tool into the target mass, in particular the region overlying the targeted mass. The anesthesia is preferably a local anesthesia such as a topically applied anesthesia gel, local anesthesia injection, or other suitable numbing agent. However, the anesthesia can include any suitable substance and/or technique. Step S270 can further comprise retracting the biopsy tool from the target mass.

The method 200 can further comprise Step 280, which recites monitoring advancement and/or placement of the biopsy tool into the target mass. Step S280 provides a safety protocol during advancement of the biopsy tool, and can further function to facilitate sampling of from the target mass during a biopsy procedure. As such, Step S280 can comprise monitoring the tissue sampling process, generating data that can facilitate adjustment of biopsy procedure parameters (e.g., rotation and/or depth of the biopsy tool) to better sample areas of the target mass. Additionally, Step S280 can comprise additional imaging post biopsy. For example, a marking clip can be placed post-biopsy, and imaging of the marking clip can be performed to mark the a specified area of the volume of tissue for subsequent imaging localization in the future or by other modalities.

As a person skilled in the art will recognize from the previous detailed description and from the FIGURES and claims, modifications and changes can be made to the preferred embodiments of the method 200 without departing from the scope of the method 200.

### Example Implementation of the Preferred System and Method

The following example implementations of the system 100 and method 200 are for illustrative purposes only, and should not be construed as definitive or limiting of the scope of the claimed invention.

In one example, a patient lies prone with the breast extended through an appropriately sized hole within a thin, pliable membrane that allows the pendulous breast to fully expose the underlying chest wall and the axilla. The axial length of the pendulous breast, noted in water during a prior ultrasound tomographic scan, is used to limit the excursion of the breast in air. A movable base provides this support by gently lifting the pendulous breast up to the lower edge of a multiplanar transducer array, thereby confining the breast length to the height of the transducer array. The overall transducer array includes a “wall” of modular stacked transducer subarrays that is eight transducer arrays tall (i.e. total of 8192 elements with current processing capacity), thereby providing for a “2.5”-dimensional scannable volume. Each of the 1024 transducer elements within each transducer subarrays is approximately twenty-two millimeters tall and provides a centrally focused three millimeter acoustic beam height. Furthermore, the transducer subarrays are configured to provide a coned-beam imaging format. The movable fixation plate additionally functions as a reflector plate to enable acoustic data related to acoustic reflection, acoustic speed, and acoustic attenuation to be generated.

The overlying skin is prepared for a sterile fixation plate, to be moved, such that the fixation plate gently but firmly compresses the breast, to hold the breast in place for minimal distortion during needle insertion. Mass localization within the three-dimensional volume scanned by the multiplanar transducer subarrays is then compared to the original mass localization seen on the initial ultrasound tomographic scan in water. The insert for either fine needle or large core biopsy is placed into the appropriate square within the aperture of a guiding module that is substantially aligned with the targeted mass. The orientation of the guiding module is such that a long-axis of breast compression/fixation can be used to perform the biopsy procedure. The overlying skin is anesthetized and the selected needle, inserted to the required depth to reach the targeted mass, according to direct visualization provided by the multiplanar transducer array, an example of which is shown in FIGS. 11A-11D with a target mass more clearly located in FIG. 11C. The inserted needle implants a hookwire, fires an automated core biopsy, or obtains vacuum-assisted biopsy samples. Finally, the biopsy needle is retracted and the patient is stabilized using a suitable post-biopsy procedure.

The system 100 and method 200 of the preferred embodiment and variations thereof can be embodied and/or implemented at least in part as a machine configured to receive a computer-readable medium storing computer-readable instructions. The instructions are preferably executed by computer-executable components preferably integrated with the system and one or more portions of the processor 140 and/or the controller 150. The computer-readable medium can be stored on any suitable computer-readable media such as RAMs, ROMs, flash memory, EEPROMs, optical devices (CD or DVD), hard drives, floppy drives, or any suitable device. The computer-executable component is preferably a general or application specific processor, but any suitable dedicated hardware or hardware/firmware combination device can alternatively or additionally execute the instructions.

The FIGURES illustrate the architecture, functionality and operation of possible implementations of systems, methods and computer program products according to preferred embodiments, example configurations, and variations thereof. In this regard, each block in the flowchart or block diagrams can represent a module, segment, or portion of code, which comprises one or more executable instructions for implementing the specified logical function(s). It should also be noted that, in some alternative implementations, the functions noted in the block can occur out of the order noted in the FIGURES. For example, two blocks shown in succession can, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As a person skilled in the art will recognize from the previous detailed description and from the figures and claims, modifications and changes can be made to the preferred embodiments of the invention without departing from the scope of this invention defined in the following claims.

What is claimed is:

1. A system for performing an image-guided biopsy of a target mass of a volume of tissue with a biopsy tool, the system comprising:
a transducer array comprising a set of ultrasound emitters configured to emit acoustic waveforms toward the volume of tissue, and a set of ultrasound receivers configured to generate a set of acoustic data based upon acoustic waveforms received from the volume of tissue, wherein the transducer array is configured to communicate with a processor configured to enable determination of a location of the target mass from the set of acoustic data;
a base proximate to the transducer array;
a fixation plate coupled to the base and cooperating with the base and the transducer array to at least partially define an adjustable receiving space configured to receive the volume of tissue; and
a guiding module coupled to the base and defining an aperture configured to align the biopsy tool with the location of the target mass.

2. The system of claim 1, wherein the transducer array defines a boundary of a receiving space for the volume of tissue.

3. The system of claim 1, wherein the transducer array comprises a stack of transducer subarrays providing more than one imaging plane.

4. The system of claim 3, wherein the transducer array comprises orthogonally oriented transducer elements, configured to provide orthogonal imaging planes.

5. The system of claim 1, wherein the transducer array is configured to emit a three-dimensional coned beam toward the volume of tissue.

6. The system of claim 1, wherein the transducer array is configured to communicate with a processor comprising at least one multiplexer.

7. The system of claim 1, wherein the transducer array is configured to generate a set of acoustic data characterizing at least one of acoustic reflection, acoustic speed, and acoustic attenuation.

8. The system of claim 1, wherein the fixation plate is physically coextensive with a reflector plate configured to reflect acoustic signals from the transducer array.

9. The system of claim 1, wherein the fixation plate comprises a second transducer array, configured to oppose the transducer array.

10. The system of claim 8, wherein a surface of the transducer array is oriented opposite to a surface of the fixation plate and wherein the guiding module comprises a surface oriented orthogonal to the surface of the transducer array and the surface of the fixation plate, such that a biopsy tool can be guided into the volume of tissue using a long axis of tissue stabilization approach.

11. The system of claim 1, wherein the fixation plate is physically coextensive with the guiding module.

12. The system of claim 1, wherein at least one of the base, the transducer array, the fixation plate, and the guiding module is coupled to an actuator configured to adjust the adjustable receiving space.

13. A method for performing an image-guided biopsy of a target mass of a volume of tissue, the method comprising:
receiving the volume of tissue in a receiving space defined at least partially by a transducer array and a fixation plate;
stabilizing the volume of tissue within the receiving space;
at the transducer array, emitting acoustic waveforms toward the volume of tissue;
generating a set of acoustic data based upon acoustic waveforms received from the volume of tissue;
rendering an image defining a location of the target mass, based upon the set of acoustic data;
at a guiding module, aligning a biopsy tool with the location of the target mass; and
advancing the biopsy tool into the target mass.

14. The method of claim 13, further comprising performing an ultrasound tomographic planning scan of the volume of tissue, and measuring a characteristic of the volume of tissue, wherein the characteristic comprises a dimension of the volume of tissue.

15. The method of claim 14, wherein performing an ultrasound tomographic planning scan and measuring a characteristic of the volume of tissue comprise manipulating the volume of tissue into a stabilized configuration, and stabilizing the volume of tissue comprises manipulating the volume of tissue into the stabilized configuration.

16. The method of claim 13, wherein stabilizing the volume of tissue within the receiving space comprises actuating at least one of the transducer array and the fixation plate.

17. The method of claim 13, wherein emitting acoustic waveforms toward the volume of tissue comprises emitting a three-dimensional coned-beam toward the volume of tissue.

18. The method of claim 13 wherein generating a set of acoustic data comprises generating data obtained from multiple imaging planes using a coned-beam imaging format.

19. The method of claim 13, wherein generating a set of acoustic data comprises generating data obtained from opposed transducer elements, in order to generate direct transmission data.

20. The method of claim 18, wherein rendering an image comprises rendering a 2.5-dimensional ultrasound image in substantially real time.

21. The method of claim 13, wherein advancing the biopsy tool into the target mass comprises advancing the biopsy tool into the target mass long a long axis of tissue stabilization.

22. The method of claim 13, further comprising monitoring advancement of the biopsy tool into the target mass, and placing a marking clip into the target mass.
